(19) United States
(12) Patent Application Publication
(10) Pub. No.: US 2010/0303786 A1
(43) Pub. Date: Dec. 2, 2010

(54) Stabilisation of Liquid-Formulated Factor VII(A) Polypeptides by Aldehyde-Containing Compounds

(75) Inventors: Florencio Zaragoza Dörwald, Visp (CH); Henning Ralf Stennicke, Kokkedal (DK); Ole Hvilsted Olsen, Bronshoj (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk Health Care AG, Zurich (CH)

(21) Appl. No.: 12/743,612

(22) PCT Filed: Nov. 21, 2008
(86) PCT No.: PCT/EP08/65959

§ 371 Date: Jul. 29, 2010

Related U.S. Application Data
(60) Provisional application No. 61/010,608, filed on Jan. 10, 2008.

(30) Foreign Application Priority Data
Nov. 22, 2007 (EP) 07121310.2

(51) Int. Cl. A61K 38/48; A61P 7/04

(52) U.S. Cl. 424/94.3

(57) ABSTRACT

The present invention is directed to liquid, aqueous pharmaceutical compositions stabilised against chemical and/or physical degradation containing Factor VII polypeptides, and methods for preparing and using such compositions, as well as vials containing such compositions, and the use of such compositions in the treatment of a Factor VII-responsive syndrome. The main embodiment is represented by a liquid, aqueous pharmaceutical composition comprising at least 0.01 mg/mL of a Factor VII polypeptide (i); a buffering agent (ii) suitable for keeping pH in the range of from about 4.0 to about 9.0; and at least one stabilising agent (iii) comprising a R—CHO motif, e.g. Benzaldehyde, 3-hydroxybenzaldehyde, 4-hydroxybenzaldehyde, or 5-formyl-4-methylimidazole.

### FIELD OF THE INVENTION

The present invention relates to liquid, aqueous pharmaceutical compositions containing Factor VII(a) polypeptides; methods for preparing and using such compositions; containers containing such compositions and the use of such compositions for the treatment of a Factor VII(a)-responsive disorder. More particularly, the invention relates to liquid compositions stabilised against chemical and/or physical degradation.

### BACKGROUND OF THE INVENTION

Blood clotting Factor VIIa (FVIIa) has proven to be an important therapeutic agent for the treatment of blood clotting disorders such as haemophilia A, haemophilia B, Glanzmann's thrombasthenia and FVII(a) deficiency. It is also used to enhance blood coagulation in humans that are subject to life-threatening, diffuse or surgically inaccessible bleedings but who otherwise do not have a blood clotting disorder. The current commercially available, recombinant Factor VIIa formulation NovoSeven® (Novo Nordisk A/S, Denmark), is presented as a vial (about 3.0 mL container volume) containing a freeze-dried cake of 1.2 mg recombinant human Factor VIIa, 5.84 mg NaCl, 2.94 mg CaCl₂, 2 H₂O, 2.64 mg GlyGly, 0.14 mg polysorbate 80, and 60.0 mg mannitol. This product is reconstituted to pH 5.5 by 2.0 mL water for injection (WFI) immediately prior to use, thus yielding a FVIIa concentration of about 0.6 mg/mL.

The decision to either maintain a manufactured protein drug in a liquid, or to freeze-dry it, is usually based on the stability of the protein in those two forms. Protein stability can be affected inter alia by such factors as ionic strength, pH, temperature, repeated cycles of freezing and thawing, exposure to shear forces and the nature of the protein itself. Some of the active protein may be lost as a result of physical instability, resulting in denaturation and aggregation (both soluble and insoluble aggregate formation), as well as chemical instability, resulting in for example, hydrolysis, deamidation, and oxidation; to name just a few. For a general review of the stability of protein pharmaceuticals, see, for example, Manning, et al., Pharmaceutical Research 6:903-918 (1989).

Whilst the possible occurrence of protein instability is widely appreciated, it is impossible to predict the particular instability-related problems of a particular protein. Instability can result in the formation of a protein by-product, or derivative, that has lowered activity, increased toxicity, and/or increased immunogenicity. Furthermore, post-translational modifications such as the gamma-carboxylation of certain glutamic acid residues in the N-terminus, or the addition of carbohydrate side chains, provide potential sites of modification during storage.

Furthermore, liquid formulations of serine proteases, such as Factor VII(a) polypeptides, are subject to degradation by autolysis because they themselves are both biological enzymes and substrates. Factors II(a), VII(a), IX(a), X(a) and XI(a) are four such examples of serine proteases. Formulating a protease such as a FVII polypeptide is a major challenge to the pharmaceutical industry because FVII(a) polypeptides readily cleave other FVII polypeptides in the same formulation, rendering them inactive. In liquid formulations, FVII(a) polypeptides can autolyse within a period of a few hours and the problem is particularly acute when the concentration of FVII(a) polypeptide is high. Therefore, in creating a liquid formulation of a FVII(a) polypeptide, autolysis is the greatest hurdle to be overcome.

Thus, the safety and efficacy of any protein composition is directly related to its stability. Maintaining protein stability in a liquid requires a different approach to maintaining stability in its lyophilized form because of the highly increased potential for molecular motion and therefore increased probability of molecular interactions. Maintaining stability in a concentrated solution is also different from the above, because of the propensity for aggregate formation at increased protein concentrations.

When developing a liquid composition, many factors are taken into consideration. Obtaining short-term (less than six months) liquid stability generally requires avoiding gross structural changes, such as denaturation and aggregation. These processes are described in the literature for a number of proteins, and many examples of stabilizing agents exist. It is well-known that an agent effective in stabilizing one protein actually acts to destabilize another. Once the protein has been stabilized against gross structural changes, developing a liquid composition for long-term stability (e.g., greater than six months) depends on further stabilizing the protein from types of degradation specific to that protein. More specific types of degradation may include, for example, disulfide bond scrambling, oxidation of certain residues, deamidation, cyclization. Although it is not always possible to pinpoint the individual degradation species, assays are developed to monitor subtle changes so as to monitor the ability of specific excipients to uniquely stabilize the protein of interest.

It is also desirable that the pH of the composition is in a physiologically suitable range upon injection/infusion. For a general review of protein compositions, see, for example, Cleland et al.: The development of stable protein compositions: A closer look at protein aggregation, deamidation and oxidation, Critical Reviews in Therapeutic Drug Carrier Systems 1993, 10(4): 307-377; and Wang et al., Parenteral compositions of proteins and peptides: Stability and stabilizers, Journal of Parenteral Science and Technology 1988 (Supplement), 42 (2S).

Factor VIIa undergoes several degradative pathways, especially aggregation (dimerisation), oxidation and autolytic cleavage (clipping of the peptide backbone or “heavy chain degradation”). Furthermore, precipitation may occur. Many of these reactions can be slowed significantly by removal of water from the protein.

There are several advantages associated with the use of a preserved, liquid formulation rather than a freeze-dried cake that is reconstituted with WFI immediately prior to injection. Most notably, a preserved liquid is much more convenient to use than a freeze-dried product. The development of a liquid composition of a Factor VIIa polypeptide could eliminate reconstitution errors, thereby increasing dosing accuracy; as well as simplifying the use of the product clinically, thereby increasing patient compliance. Ideally, compositions of a Factor VIIa polypeptide should be stable for more than 6 months over a wide range of protein concentrations, which may in turn provide flexibility in terms of the route of administration. Generally, more highly concentrated solutions allow for the administration of lower volumes, which may provide an opportunity for parenteral administration other than intravenous. Liquid compositions can thus have many advantages over freeze-dried products with regard to ease of administration and use.

Currently, no liquid-formulated FVIIa product is commercially available. It is an objective of this invention to provide a liquid Factor VII(a) polypeptide pharmaceutical composition which is suitable for both storage and delivery and in which the amount of chemical and/or physical degradation products is physiologically acceptable.

### BRIEF DESCRIPTION OF THE DRAWINGS

FIG. 1: Effect of aldehydes on FVIIa amidolytic activity. All of the aldehydes examined inhibit amidolytic activity.

FIG. 2: Effect of aldehydes on factor VIIa melting point (denaturation temperature). 3-hydroxybenzaldehyde, 4-hydroxybenzaldehyde and benzaldehyde affect the denaturation temperature at high concentrations. 5-formyl-4-methylimidazol does not affect the denaturation temperature until a concentration of about 100 mM.

FIG. 3: Fragmentation of FVIIa after 14 days in aldehyde solutions All of the aldehydes examined reduce the amount of FVIIa fragmentation.

FIG. 4: Protease activity recovery upon dilution of a FVIIa/aldehyde formulation. Protease activity recovery is compared to the protease activity of a fresh sample of a FVIIa solution that does not contain an aldehyde.

FIG. 5: FVIIa fragmentation after 4 weeks in benzaldehyde solutions of different concentration. The percentage of FVIIa fragmentation decreases as the concentration of benzaldehyde is increased.

### SUMMARY OF THE INVENTION

The present inventors have created liquid pharmaceutical compositions of Factor VII(a) polypeptides that exhibit improved stability. In these compositions, the Factor VII(a) polypeptides are formulated with at least one stabilising agent comprising the R—CHO motif. The inventors anticipate that the at least one stabilising agent comprising said R—CHO motif can also be used to stabilise the other serine proteases, namely FII(a), FIX(a), FX(a), FXI(a) protein C and protein S.

Thus, one aspect of the present invention relates to a liquid, aqueous pharmaceutical composition comprising at least 0.01 mg/mL of a Factor VII(a) polypeptide (i); a buffering agent (ii) suitable for keeping pH in the range of from about 4.0 to about 9.0; and at least one stabilising agent (iii) comprising a stabilizing agent of general formula R—CHO, wherein R represents aryl, optionally substituted once or several times with hydroxy, C₁₋₆-alkoxy, carboxyl, hydroxymethyl, C₁₋₆-alkyl, C₂₋₆-alkenyl, cyano, or halogen; heteroaryl, optionally substituted once or several times with hydroxy, C₁₋₆-alkoxy, carboxyl, hydroxymethyl, C₁₋₆-alkyl, C₂₋₆-alkenyl, cyano, or halogen; R¹R²R³C—, wherein R¹, R², and R³ independently represent hydrogen, C₁₋₆-alkyl, C₂₋₆-alkenyl, hydroxy, hydroxymethyl, amino, dimethylamino, methoxycarbonylamino, ethoxycarbonylamino, or dimethylaminomethyl; or R⁴C(═O)—, wherein R⁴ represents methyl, ethyl, hydroxy, amino, C₁₋₆-alkoxy, C₁₋₆-alkylamino, or C₂₋₆-dialkylamino.

A second aspect of the present invention relates to a method for preparing a liquid, aqueous pharmaceutical composition of a Factor VII(a) polypeptide, comprising the step of providing the Factor VII(a) polypeptide (i) at a concentration of at least 0.01 mg/mL in a solution comprising a buffering agent (ii) suitable for keeping pH in the range of from about 4.0 to about 9.0; and at least one stabilising agent (iii) comprising a R—CHO motif, wherein R represents aryl, optionally substituted once or several times with hydroxy, C₁₋₆-alkoxy, carboxyl, hydroxymethyl, C₁₋₆-alkyl, C₂₋₆-alkenyl, cyano, or halogen; heteroaryl, optionally substituted once or several times with hydroxy, C₁₋₆-alkoxy, carboxyl, hydroxymethyl, C₁₋₆-alkyl, C₂₋₆-alkenyl, cyano, or halogen; R¹R²R³C—, wherein R¹, R², and R³ independently represent hydrogen, C₁₋₆-alkyl, C₂₋₆-alkenyl, hydroxy, hydroxymethyl, amino, dimethylamino, methoxycarbonylamino, ethoxycarbonylamino, or dimethylaminomethyl; or R⁴C(═O)—, wherein R⁴ represents methyl, ethyl, hydroxy, amino, C₁₋₆-alkoxy, C₁₋₆-alkylamino, or C₂₋₆-dialkylamino.

A third aspect of the present invention relates to the liquid, aqueous pharmaceutical composition for use as a medicament.

A fourth aspect of the present invention relates to the use of the liquid, aqueous pharmaceutical composition for the preparation of a medicament for treating a Factor VII(a)-responsive disorder.

A fifth aspect of the present invention relates to a method for treating a Factor VII(a)-responsive disorder, the method comprising administering to a subject in need thereof an effective amount of the liquid, aqueous pharmaceutical composition.

A sixth aspect of the present invention relates to an air-tight container containing the liquid, aqueous pharmaceutical composition and, optionally, an inert gas.

### DETAILED DESCRIPTION OF THE INVENTION

There are two known, naturally occurring conformations of the serine protease Factor VIIa, known as inactive FVIIa and active FVIIa. Inactive FVIIa is the intravenous protease which has been cleaved at the peptide bond preceding position 153 {16} (trypsinogen numbering in brackets) by any one of Factors IXa, Xa or FVIIa but which still maintains a three-dimensionally inactive conformation. Active FVIIa represents the three-dimensionally active conformation which, in vivo, is primarily induced by tissue factor (TF) upon vessel wall injury. In trypsin the newly formed N-terminus spontaneously inserts into a cavity, termed the activation pocket, resulting in the formation of a salt bridge to Asp343 {194}. The formation of the salt bridge leads to maturation of the catalytic apparatus i.e. the substrate binding cleft and the oxyanion hole. This same mechanism applies to all of the serine proteases but, upon activation, FVIIa has a very low catalytic activity due to poor N-terminal insertion. TF allosterically facilitates this process and markedly accelerates the activity of FVIIa towards its physiological substrates. However, even without the presence of TF, an equilibrium exists between intravenous inactive FVIIa and active FVIIa, such that approximately 95% FVIIa is normally in the inactive conformation and approximately 5% FVIIa is normally in the active conformation.

It is very difficult but most desirable that the inactive FVIIa conformation is maintained in the pharmaceutical formulation, with its conversion to active FVIIa harnessed until its release into the blood stream.

It is known that some aldehydes react reversibly with amines to form imines (Schiff bases) (Dohno et al., J. Am. Chem. Soc. 2005, 127, 16681-16684; Ge et al., J. Agric. Food Chem. 1997, 45, 1619-1623):

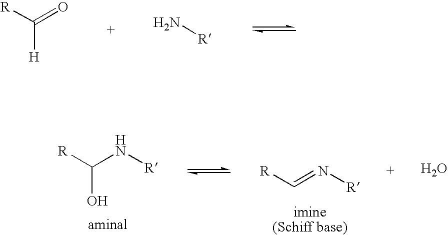

In terms of the present invention, R′—NH₂ represents the amine terminal of the inactive FVIIa polypeptide and R—CHO represents an aldehyde-containing stabilising agent. As only the unprotonated amine [terminal] can add to the aldehyde and because the amine and the imine (Schiff base) are both bases but have different basicities, the rate of imine formation and the equilibrium concentrations of all components depend on the pH of the reaction mixture (Sayer et al., J. Am. Chem. Soc. 1973, 95, 4277-4287; Garcia del Vado et al., J. Mol. Catal. A: Chem. 1996, 111, 193-201).

Usually, Schiff bases are prepared under slightly acidic conditions, e.g. in the presence of acetic acid. However, only the unprotonated form of the amine will react with aldehydes. Thus, under very strongly acidic conditions the concentration of free amino groups will become rate-limiting (i.e. aminal-formation becomes the rate-determining, slowest step) and the rate of imine-formation will drop again. If various different amines are present in the reaction mixture, under slightly acidic conditions the amine of lowest basicity will be converted into an aminal or imine first, because a higher proportion of the amine of lower basicity will be present in its unprotonated form.

In proteins, two types of amino groups are usually present: the N-terminal amino group and the side-chain amino groups of lysines. Because the N-terminal amino group is located close to a carbonyl group, it is less basic than the side chain amino groups of lysines. Upon addition of an aldehyde to a protein, this slight difference of basicity leads to a faster imine formation with the N-terminal amino group than with the side-chain amino groups.

With an excess of aldehyde most amino groups will either be present as aminal or as imine, and almost no free amino groups will remain. On the other hand, if an imine is dissolved in a large amount of water, and other amines are present, hydrolysis of the imine can occur to liberate the amine-component of the original imines.

The inventors of the present invention expect that if one equivalent of an aldehyde is added to a protein under slightly acidic conditions then an aminal or imine will be formed mainly with the N-terminal amino group. Accordingly, because the N-terminal amino group in FVIIa is crucial for the three-dimensional activation of the protease, the addition of aldehydes capable of reversible imine-formation will enhance the stability of aqueous solutions of FVIIa. Upon injection into a mammal of such a stabilized solution, i.e. upon dilution and in the presence of numerous other amines, the imine of FVIIa will be hydrolyzed, and the activity of this protease will be regained.

Thus, the present invention encompasses the use of low molecular weight, aldehyde-containing compounds which reversibly bind the amine terminal of the FVIIa polypeptide. At high concentrations, the aldehyde-containing compounds bind surface accessible free amines, particularly the amine of the N-terminal, and therefore prevent the FVIIa polypeptide's amine terminal from inserting into its activation pocket. Upon injection into the bloodstream, however, the pharmaceutical formulation is diluted. Therefore, the FVIIa polypeptide's amine terminal dissociates from the aldehyde and is able to insert into its activation pocket, resulting in a FVIIa polypeptide which can adopt an active three-dimensional conformation.

In a primary screen four compounds containing aldehydes have been tested: Benzaldehyde, 3-hydroxybenzaldehyde, 4-hydroxybenzaldehyde, and 5-formyl-4-methylimidazole.

As mentioned above, the present invention resides in the development of a novel stabilised liquid, aqueous pharmaceutical composition comprising a Factor VII(a) polypeptide. More specifically, the liquid, aqueous pharmaceutical composition comprises at least 0.01 mg/mL of a Factor VII polypeptide (i); a buffering agent (ii) suitable for keeping pH in the range of from about 4.0 to about 9.0; and at least one stabilising agent (iii) comprising a R—CHO motif, wherein R represents aryl, optionally substituted once or several times with hydroxy, C₁₋₆-alkoxy, carboxyl, hydroxymethyl, C₁₋₆-alkyl, C₂₋₆-alkenyl, cyano, or halogen; heteroaryl, optionally substituted once or several times with hydroxy, C₁₋₆-alkoxy, carboxyl, hydroxymethyl, C₁₋₆-alkyl, C₂₋₆-alkenyl, cyano, or halogen; R¹R²R³C—, wherein R¹, R², and R³ independently represent hydrogen, C₁₋₆-alkyl, C₂₋₆-alkenyl, hydroxy, hydroxymethyl, amino, dimethylamino, methoxycarbonylamino, ethoxycarbonylamino, or dimethylaminomethyl; or R⁴C(═O)—, wherein R⁴ represents methyl, ethyl, hydroxy, amino, C₁₋₆-alkoxy, C₁₋₆-alkylamino, or C₂₋₆-dialkylamino.

The term “C₁₋₆-alkyl” is intended to encompass acyclic and cyclic saturated hydrocarbon residues which have 1-6 carbon atoms and which can be linear or branched. Particular examples are methyl, ethyl, n-propyl, isopropyl, cyclopropyl, n-butyl, isobutyl, sec-butyl, tert-butyl, cyclopropylmethyl, n-pentyl, isopentyl, n-hexyl, etc. Similarly, the term “C₁₋₄-alkyl” encompasses acyclic and cyclic saturated hydrocarbon residues which have 1-4 carbon atoms and which can be linear or branched.

Similarly, the term “C₂₋₆-alkenyl” is intended to encompass acyclic and cyclic hydrocarbon residues which have 2-6 carbon atoms and comprise one unsaturated bond, which can be linear or branched. Examples of C₂₋₆-alkenyl groups are vinyl, allyl, but-1-en-1-yl, but-2-en-1-yl, pent-1-en-1-yl, and hex-1-en-1-yl.

The term “optionally substituted” in connection with C₁₋₆-alkyl and C₂₋₆-alkenyl groups is intended to denote that the group in question may be substituted one or several times, preferably 1-3 times, with group(s) selected from the group consisting of hydroxy, C₁₋₆-alkoxy (i.e. C₁₋₆-alkyl-oxy), C₂₋₆-alkenyloxy, oxo (forming a keto or aldehyde functionality), aryl, aryloxy, arylcarbonyl, heterocyclyl, heterocyclyloxy, heterocyclylcarbonyl, amino, mono- and di(C₁₋₆-alkyl)amino, halogen, where any aryl and heterocyclyl may be substituted as specifically described below for optionally substituted aryl and heterocyclyl.

“Halogen” includes fluoro, chloro, bromo, and iodo.

When used herein, the term “aryl” is intended to denote a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl, among which phenyl is a preferred example.

The terms “heterocyclyl” and “heterocyclic ring” is intended to denote a saturated, partially unsaturated, partially aromatic or fully aromatic carbocyclic ring or ring system where one or more of the carbon atoms have been replaced with heteroatoms, e.g. nitrogen (═N— or —NH), sulphur (—S—), and/or oxygen (—O—) atoms. Examples of such heterocyclyl groups are oxazolyl, oxazolinyl, oxazolidinyl, isoxazolyl, isoxazolinyl, isoxazolidinyl, oxadiazolyl, oxadiazolinyl, oxadiazolidinyl, thiazolyl, iso-thiazolyl, pyrrolyl, pyrrolinyl, pyrrolidinyl, imidazolyl, imidazolinyl, imidazolidinyl, pyrazolyl, pyridinyl, pyrazinyl, pyridazinyl, piperidinyl, coumaryl, furyl, quinolyl, benzothiazolyl, benzotriazolyl, benzodiazolyl, benzoxozolyl, diazolyl, diazolinyl, diazolidinyl, triazolyl, triazolinyl, triazolidinyl, tetrazol, etc. Preferred heterocyclyl groups are 5-, 6- or 7-membered monocyclic groups such as isoxazolyl, isoxazolinyl, oxadiazolyl, oxadiazolinyl, pyrrolyl, pyrrolinyl, diazolyl, diazolinyl, triazolyl, triazolinyl, imidazolyl, imidazolinyl, etc.

In connection with the terms “aryl”, “heterocyclyl” and “heterocyclic ring”, the term “optionally substituted” is intended to denote that the group in question may be substituted one or several times, preferably 1-3 times, with group(s) selected from hydroxy (which when present in an enol system may be represented in the tautomeric keto form), C₁₋₆-alkyl, C₂₋₆-alkenyl, phenyl, benzyl, C₁₋₆-alkoxy, oxo (which may be represented in the tautomeric enol form), carboxy, C₁₋₆-alkoxycarbonyl, C₁₋₆-alkylcarbonyl, amino, mono- and di(C₁₋₆-alkyl)amino, dihalogen-C₁₋₄-alkyl, trihalogen-C₁₋₄-alkyl, and halogen. The most typical examples of substituents are hydroxyl, C₁₋₄-alkyl, phenyl, benzyl, C₁₋₄-alkoxy, oxo, amino, mono- and dimethylamino and halogen.

It is believed that the R—CHO motif is particularly important for the stabilising effect of the stabilising agent, wherein R represents aryl, optionally substituted once or several times with hydroxy, methoxy, carboxyl, hydroxymethyl, methyl, ethyl, isopropyl, or cyano; heteroaryl, optionally substituted once or several times with hydroxy, methoxy, carboxyl, hydroxymethyl, methyl, ethyl, isopropyl, or cyano; R¹R²R³C—, wherein R¹, R², and R³ independently represent hydrogen, methyl, hydroxy, hydroxymethyl, amino, dimethylamino, methoxycarbonylamino, ethoxycarbonylamino, or dimethylaminomethyl; or R⁴C(═O)—, wherein R⁴ represents methyl, ethyl, hydroxy, amino, methoxy, ethoxy, methylamino or dimethylamino.

In another embodiment, R of the R—CHO motif represents phenyl, optionally substituted once or several times with hydroxy, carboxyl, or hydroxymethyl; imidazolyl, optionally substituted once or several times with hydroxy, carboxyl, hydroxymethyl, or methyl; R¹R²R³C—, wherein R¹, R², and R³ independently represent hydrogen, methyl, hydroxy, hydroxymethyl, methoxycarbonylamino, ethoxycarbonylamino, or dimethylaminomethyl; or R⁴C(═O)—, wherein R⁴ represents methyl, ethyl, hydroxy, amino, methoxy, ethoxy, methylamino or dimethylamino.

In one particular embodiment, at least one of R¹ and R² is hydrogen.

In another embodiment, the stabilising agent (iii) is at least one selected from the group consisting of benzaldehyde, acetaldehyde, pivalic aldehyde (trimethylacetaldehyde), isobutyraldehyde, 4-methyl-5-formylimidazole, 3-hydroxybenzaldehyde, hydroxypivaldehyde, 3-dimethylamino-2,2-dimethylpropionaldehyde, salicylaldehyde, 4-hydroxybenzaldehyde, 2,3-dihydroxybenzaldehyde, 2,4-dihydroxybenzaldehyde, 2,5-dihydroxybenzaldehyde, 3,4-dihydroxybenzaldehyde, 2-carboxybenzaldehyde, 4-carboxybenzaldehyde, 2-formylpyridine, 3-formylpyridine, 4-formylpyridine, 2-formylimidazole, 4-formylimidazole, and 2-hydroxymethyl-4-formylimidazole.

The compounds of the present invention may have one or more asymmetric centres and unless otherwise indicated it is intended that stereoisomers (optical isomers), as separated, pure or partially purified stereoisomers or racemic mixtures thereof are included in the scope of the invention.

The concentration of the stabilising agent (or agents) (iii) is typically at least 1 μM. The desirable (or necessary) concentration typically depends on the selected stabilising agent (or agents), more specifically on the binding affinity of the selected stabilising agent to the Factor VII(a) polypeptide.

In different embodiments, the stabilising agent (iii) is present in a concentration of at least 5 μM, at least 10 μM, at least 20 μM, at least 50 μM, at least 100 μM, at least 150 μM, at least 250 μM, at least 500 μM, at least 1 mM, at least 2 mM, at least 4 mM, at least 5 mM, at least 8 mM, at least 9 mM, at least 10 mM, at least 15 mM, at least 20 mM, such as, e.g., in the range of 1-10000 μM, 10-10000 μM, 20-10000 μM, 50-10000 μM, 10-5000 μM, 10-2000 μM, 20-5000 μM, 20-2000 μM, 50-5000 μM, 0.1-100 mM, 0.1-75 mM, 0.1-50 mM, 0.1-10 mM, 0.2-75 mM, 0.2-50 mM, 0.2-20 mM, 0.5-75 mM, 0.5-50 mM, 0.5-100 mM, 0.5-200 mM, 1-200 mM, 150-200 mM, 1-100 mM, 5-100 mM, 10-100 mM or 5-80 mM.

In one embodiment, the stabilising agent (iii) is aldehyde selected from Benzaldehyde, 3-hydroxybenzaldehyde, 4-hydroxybenzaldehyde, and 5-formyl-4-methylimidazole; and the concentration of said agent is at least 1 mM, such as, e.g., at least 2 mM, although it is envisaged that substituted aldehydes may be more potent for what reason they can be added in lower concentrations.

In one embodiment, the stabilizing agent (iii) is benzaldehyde. In a further embodiment, (iii) is benzaldehyde in a concentration of about 0.5-100 mM, such as about 1-100 mM; such as about 5-100 mM; such as about 10-100 mM; such as about 5-80 mM.

In one embodiment, the stabilizing agent (iii) is 3-hydroxybenzaldehyde.

In one embodiment, the stabilizing agent (iii) is 4-hydroxybenzaldehyde.

In one embodiment, the stabilizing agent (iii) is 5-formyl-4-methylimidazole. In a further embodiment (iii) is 5-formyl-4-methylimidazole in a concentration of about 0.5-200 mM; such as about 1-200 mM; such as about 150-200 mM.

In various embodiments, the molar ratio between the stabilising agent (iii) and FVII(a) polypeptide (agent (iii):FVII(a)) is: above 0.1, above 0.5, above 1, above 2, above 5, above 10, above 25, above 100, above 250, above 1000, above 2500, or above 5000, such as, e.g., in the range of 0.1-10000, 0.1-5000, 0.1-2500, 0.1-1000, 0.1-250, 0.1-100, 0.1-25, 0.1-10, 0.5-10000, 0.5-5000, 0.5-2500, 0.5-1000, 0.5-250, 0.5-100, 0.5-25, 0.5-10, 1-10000, 1-5000, 1-2500, 1-1000, 1-250, 1-100; 1-25; 1-10, 10-10000, 10-5000, 10-250, 1000-10000, or 1000-5000.

The desirable concentration typically depends on the selected stabilising agent (or agents), more specifically on the binding affinity of the selected agent to the Factor VII(a) polypeptide.

The biological effect of the pharmaceutical composition is mainly ascribed to the presence of the Factor VII(a) polypeptide, although other active ingredients may be included in combination with the Factor VII(a) polypeptide.

As used herein, the terms “Factor VII(a) polypeptide” or “FVII(a) polypeptide” means any protein comprising the amino acid sequence 1-406 of wild-type human Factor VIIa (i.e., a polypeptide having the amino acid sequence disclosed in U.S. Pat. No. 4,784,950), amino acid sequence variants thereof as well as Factor VII(a) derivatives and Factor VII(a) conjugates of any FVII(a) wild-type or FVII(a) sequence variant. This includes FVII(a) variants, Factor VII(a)-related polypeptides, Factor VII(a) derivatives and Factor VII(a) conjugates exhibiting substantially the same or improved biological activity relative to wild-type human Factor VIIa.

As used herein, “wild type human FVIIa” is a polypeptide having the amino acid sequence disclosed in U.S. Pat. No. 4,784,950 and being in its active form.

The term “Factor VII” is intended to encompass Factor VII polypeptides in their uncleaved (zymogen) form, as well as those that have been proteolytically processed to yield their respective bioactive forms, which may be designated Factor VIIa. Typically, Factor VII is cleaved between residues 152 and 153 to yield Factor VIIa. The term “Factor VII polypeptide” also encompasses polypeptides, including variants, in which the Factor VIIa biological activity has been substantially modified or somewhat reduced relative to the activity of wild-type Factor VIIa. These polypeptides include, without limitation, Factor VII or Factor VIIa into which specific amino acid sequence alterations have been introduced that modify or disrupt the bioactivity of the polypeptide.

The biological activity of Factor VIIa in blood clotting derives from its ability to (i) bind to Tissue Factor (TF) and (ii) catalyze the proteolytic cleavage of Factor IX or Factor X to produce activated Factor IX or X (Factor IXa or Xa, respectively).

For the purposes of the invention, biological activity of Factor VII polypeptides (“Factor VII biological activity”) may be quantified by measuring the ability of a preparation to promote blood clotting, cf. Assay 3 described herein. In this assay, biological activity is expressed as the reduction in clotting time relative to a control sample and is converted to “Factor VII units” by comparison with a pooled human serum standard containing 1 unit/mL Factor VII activity. Alternatively, Factor VIIa biological activity may be quantified by (i) measuring the ability of Factor VIIa or a Factor VII-related polypeptide to produce activated Factor X (Factor Xa) in a system comprising TF embedded in a lipid membrane and Factor X. (Persson et al., J. Biol. Chem. 272:19919-19924, 1997); (ii) measuring Factor X hydrolysis in an aqueous system (“In Vitro Proteolysis Assay”, see Assay 2 below); (iii) measuring the physical binding of Factor VIIa or a Factor VII-related polypeptide to TF using an instrument based on surface plasmon resonance (Persson, FEBS Letts. 413:359-363, 1997); or (iv) measuring hydrolysis of a synthetic substrate by Factor VIIa and/or a Factor VII-related polypeptide (“In Vitro Hydrolysis Assay”, see Assay 1 below).

Factor VII variants having substantially the same or improved biological activity relative to wild-type Factor VIIa encompass those that exhibit at least about 25%, such as, e.g., at least about 50%, at least about 75% or at least about 90% of the specific activity of Factor VIIa that has been produced in the same cell type, when tested in one or more of a clotting assay (Assay 3), proteolysis assay (Assay 2), or TF binding assay as described above. Factor VII variants having substantially reduced biological activity relative to wild-type Factor VIIa are those that exhibit less than about 25%, such as, e.g., less than about 10%, or less than about 5% of the specific activity of wild-type Factor VIIa that has been produced in the same cell type when tested in one or more of a clotting assay (Assay 3), proteolysis assay (Assay 2), or TF binding assay as described above. Factor VII variants having a substantially modified biological activity relative to wild-type Factor VII include, without limitation, Factor VII variants that exhibit TF-independent Factor X proteolytic activity and those that bind TF but do not cleave Factor X.

Variants of Factor VII, whether exhibiting substantially the same or better bioactivity than wild-type Factor VII, or, alternatively, exhibiting substantially modified or reduced bioactivity relative to wild-type Factor VII, include, without limitation, polypeptides having an amino acid sequence that differs from the sequence of wild-type Factor VII by insertion, deletion, or substitution of one or more amino acids.

Non-limiting examples of Factor VII variants having substantially the same biological activity as wild-type Factor VII include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189; and FVII variants exhibiting increased proteolytic stability as disclosed in WO 02/38162 (Scripps Research Institute); FVII variants having a modified Gla-domain and exhibiting an enhanced membrane binding as disclosed in WO 99/20767 (University of Minnesota); and FVII variants as disclosed in WO 01/58935 (Maxygen ApS).

Non-limiting examples of Factor VII variants having increased biological activity compared to wild-type FVIIa include FVII variants as disclosed in WO 01/83725, WO 02/22776, WO 02/077218, WO 03/27147, WO 03/37932; WO 02/38162 (Scripps Research Institute); and FVIIa variants with enhanced activity as disclosed in JP 2001061479 (Chemo-Sero-Therapeutic Res Inst.).

Non-limiting examples of Factor VII variants having substantially reduced or modified biological activity relative to wild-type Factor VII include R152E-FVIIa (Wildgoose et al., Biochem 29:3413-3420, 1990).

Examples of Factor VII polypeptides include, without limitation, wild-type Factor VII, L305V-FVII, L305V/M306D/D309S-FVII, L3051-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, M298Q/K337A-FVII, V158D/E296V/M298Q/K337A-FVII, V158D/E296V/M298Q/L305V/K337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305V/K337A/V158T-FVII, L305V/K337A/M298Q-FVII, L305V/K337A/E296V-FVII, L305V/K337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296V/K337A-FVII, L305V/V158D/K337A/M298Q-FVII, L305V/V158D/E296V/K337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305V/K337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296V/K337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296V/K337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M 298Q-FVII, K316Q/L305V/V158T-FVII, K316Q/L305V/K337A/V158T-FVII, K316Q/L305V/K337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305V/K337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/5314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/5314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296V/K337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, Factor VIIa lacking the Gla domain; and P11Q/K33E-FVII, T106N-FVII, K143N/N145T-FVII, V253N-FVII, R290N/A292T-FVII, G291N-FVII, R315N/V317T-FVII, K143N/N145T/R315N/V317T-FVII; and FVII having substitutions, additions or deletions in the amino acid sequence from 233Thr to 240Asn, FVII having substitutions, additions or deletions in the amino acid sequence from 304Arg to 329Cys, and FVII having substitutions, deletions, or additions in the amino acid sequence Ile153-Arg223.

In some embodiments, the Factor VII polypeptide is human Factor VIIa (hFVIIa), preferably recombinantly made human Factor VIIa (rhVIIa).

In other embodiments, the Factor VII polypeptide is a Factor VII(a) sequence variant.

In some embodiments, the Factor VII polypeptide has a glycosylation different from wild-type human Factor VII.

In other embodiments, the Factor VII polypeptide is a Factor VII derivative. The term “Factor VII derivative” as used herein, is intended to designate a FVII polypeptide exhibiting substantially the same or improved biological activity relative to wild-type Factor VII, in which one or more of the amino acids of the parent peptide have been genetically and/or chemically and/or enzymatically modified, e.g. by alkylation, glycosylation, PEGylation, acylation, ester formation or amide formation or the like. This includes but is not limited to PEGylated human Factor VIIa, cysteine-PEGylated human Factor VIIa and variants thereof. Non-limiting examples of Factor VII derivatives includes glycoPEGylated FVII derivatives as disclosed in WO 03/31464 and US Patent applications US 20040043446, US 20040063911, US 20040142856, US 20040137557, US 20040132640, WO2007022512, and US 20070105755 (Neose Technologies, Inc.); FVII conjugates as disclosed in WO 01/04287, US patent application 20030165996, WO 01/58935, WO 03/93465 (Maxygen ApS) and WO 02/02764, US patent application 20030211094 (University of Minnesota); modified Factor VII polypeptides as disclosed in WO2008127702 (Catalyst Biosciences, Inc.), WO2005024006 (Novo Nordisk Health Care AG); Factor VII variants as disclosed in WO2007031559, WO2007039475 and WO2007115953 (Novo Nordisk Health Care AG), WO2004029090, WO2003037932, WO2003027147, WO02077218, WO200222776 and WO200183725 (Novo Nordisk A/S); modified glycoproteins such as those disclosed in WO08025856, WO05014035 and WO2006035057 (Novo Nordisk A/S); and conjugates such as those disclosed in WO2005014049 (Novo Nordisk A/S).

In various embodiments, e.g. those where the Factor VII polypeptide is a Factor VII-related polypeptide or a Factor VII(a) sequence variant, the ratio between the activity of the Factor VII polypeptide and the activity of native human Factor VIIa (wild-type FVIIa) is at least about 1.25, preferably at least about 2.0, or 4.0, most preferred at least about 8.0, when tested in the “In Vitro Proteolysis Assay” (Assay 2) as described in the present specification.

In some embodiments, the Factor VII polypeptides are Factor VII-related polypeptides, in particular variants, wherein the ratio between the activity of said Factor VII polypeptide and the activity of native human Factor VIIa (wild-type FVIIa) is at least about 1.25 when tested in the “In Vitro Hydrolysis Assay” (Assay 1); in other embodiments, the ratio is at least about 2.0; in further embodiments, the ratio is at least about 4.0.

In a pharmaceutical composition, it is often desirable that the concentration of the active ingredient is such that the application of a unit dose does not cause unnecessary discomfort to the patient. Thus, a unit dose of more than about 2-10 mL is often undesirable. For the purpose of the present invention, the concentration of the Factor VII polypeptide is therefore at least 0.01 mg/mL. In different embodiments, the Factor VII polypeptide is present in a concentration of about 0.01-30 mg/mL; such as about 0.1-30.0 mg/mL; such as about 0.01-20 mg/mL; such as about 0.1-20 mg/mL; such as about 10 mg/mL; such as about 15-20 mg/mL; such as 0.1-15 mg/mL; such as 10-15 mg/mL; such as 0.1-10 mg/mL; such as 0.5-5.0 mg/mL; such as 0.6-4.0 mg/mL; such as 1.0-4.0 mg/mL; such as 0.1-5 mg/mL; such as 0.1-4.0 mg/mL; such as 0.1-2 mg/mL; or 0.1-1.5 mg/mL.

Factor VIIa concentration is conveniently expressed as mg/mL or as IU/mL, with 1 mg usually representing 43,000-56,000 IU or more.

In order to render the liquid, aqueous pharmaceutical composition useful for direct parenteral administration to a mammal such as a human, it is normally required that the pH value of the composition is held within certain limits, such as from about 4.0 to about 9.0. To ensure a suitable pH value under the conditions given, the pharmaceutical composition also comprises a buffering agent (ii) suitable for keeping pH in the range of from about 4.0 to about 9.0.

The term “buffering agent” includes those agents or combinations of agents that maintain the solution pH in an acceptable range from about 4.0 to about 9.0.

In one embodiment, the buffering agent (ii) is at least one component selected from the groups consisting of acids and salts of MES, PIPES, ACES, BES, TES, HEPES, TRIS, histidine (e.g. L-histidine), imidazole, glycine, glycylglycine, glycinamide, phosphoric acid (e.g. sodium or potassium phosphate), acetic acid (e.g. ammonium, sodium or calcium acetate), lactic acid, glutaric acid, citric acid (e.g. sodium or potassium citrate), tartaric acid, malic acid, maleic acid, and succinic acid. It should be understood that the buffering agent may comprise a mixture of two or more components, wherein the mixture is able to provide a pH value in the specified range. As non-exclusive examples can be mentioned acetic acid and sodium acetate.

The concentration of the buffering agent is chosen so as to maintain the preferred pH of the solution. In various embodiments, the concentration of the buffering agent is 1-100 mM; 1-50 mM; 1-25 mM; or 2-20 mM.

In one embodiment, the pH of the composition is kept from about 4.0 to about 9.0; such as from about 4.0 to about 8.0; such as from about 5.0 to about 8.0; such as from about 4.0 to about 7.0; such as from about 5.0 to about 7.5; such as from about 5.0 and about 7.0; such as from about 5.0 to about 6.5; such as from about 5.0 to about 6.0; such as from about 5.5 to about 7.0; such as from about 5.5 to about 6.5; such as from about 6.0 to about 7.0; such as from about 6.0 to about 6.5; such as from about 6.3 to about 6.7; such as from about 5.2 to about 5.7; such as from about 4.0 to about 5.2.

In addition to the three mandatory components, the liquid, aqueous pharmaceutical composition may comprise additional components beneficial for the preparation, formulation, stability, or administration of the composition.

Hence, the pharmaceutical composition may also include a non-ionic surfactant. Surfactants (also known as detergents) generally include those agents which protect the protein from air/solution interface induced stresses and solution/surface induced stresses (e.g. resulting in protein aggregation).

Typical types of non-ionic surfactants are polysorbates, poloxamers, polyoxyethylene alkyl ethers, polyethylene/polypropylene block co-polymers, polyethyleneglycol (PEG), polyxyethylene stearates, and polyoxyethylene castor oils. Illustrative examples of non-ionic surfactants are Tween®, polysorbate 20, polysorbate 80, Brij-35 (polyoxyethylene dodecyl ether), poloxamer 188, poloxamer 407, PEG8000, Pluronic® polyols, polyoxy-23-lauryl ether, Myrj 49, and Cremophor A.

In one embodiment, the non-ionic surfactant is present in an amount of 0.005-2.0% by weight.

Also, the composition may further comprise a tonicity modifying agent (v). As used herein, the term “tonicity modifying agent” includes agents which contribute to the osmolality of the solution. The tonicity modifying agent (v) includes at least one agent selected from the group consisting of neutral salts, amino acids, peptides of 2-5 amino acid residues, monosaccharides, disaccharides, polysaccharides, and sugar alcohols. In some embodiments, the composition comprises two or more of such agents in combination.

By “neutral salt” is meant a salt that is neither an acid nor a base when dissolved in an aqueous solution.

In one embodiment, at least one tonicity modifying agent (v) is a neutral salt selected from the groups consisting of sodium salts, potassium salts, calcium salts, and magnesium salts, such as sodium chloride, potassium chloride, calcium chloride, calcium acetate, calcium gluconate, calcium laevulate, magnesium chloride, magnesium acetate, magnesium gluconate and magnesium laevulate.

In a further embodiment, the tonicity modifying agent (v) includes sodium chloride in combination with at least one selected from the groups consisting of calcium chloride, calcium acetate, magnesium chloride and magnesium acetate.

In a still further embodiment, the tonicity modifying agent (v) is at least one selected from the group consisting of sodium chloride, calcium chloride, sucrose, glucose, and mannitol.

In different embodiments, the tonicity modifying agent (v) is present in a concentration of at least 1 mM, at least 5 mM, at least 10 mM, at least 20 mM, at least 50 mM, at least 100 mM, at least 200 mM, at least 400 mM, at least 800 mM, at least 1000 mM, at least 1200 mM, at least 1500 mM, at least 1800 mM, at least 2000 mM, or at least 2200 mM.

In one series of embodiments, the tonicity modifying agent (v) is present in a concentration of 5-2200 mM, such as 25-2200 mM, 50-2200 mM, 100-2200 mM, 200-2200 mM, 400-2200 mM, 600-2200 mM, 800-2200 mM, 1000-2200 mM, 1200-2200 mM, 1400-2200 mM, 1600-2200 mM, 1800-2200 mM, or 2000-2200 mM; 5-1800 mM, 25-1800 mM, 50-1800 mM, 100-1800 mM, 200-1800 mM, 400-1800 mM, 600-1800 mM, 800-1800 mM, 1000-1800 mM, 1200-1800 mM, 1400-1800 mM, 1600-1800 mM; 5-1500 mM, 25-1400 mM, 50-1500 mM, 100-1500 mM, 200-1500 mM, 400-1500 mM, 600-1500 mM, 800-1500 mM, 1000-1500 mM, 1200-1500 mM; 5-1200 mM, 25-1200 mM, 50-1200 mM, 100-1200 mM, 200-1200 mM, 400-1200 mM, 600-1200 mM, or 800-1200 mM.

In one embodiment of the invention, at least one tonicity modifying agent (v) is an ionic strength modifying agent (v/a).

As used herein, the term “ionic strength modifying agent” includes agents which contribute to the ionic strength of the solution. The agents include, but are not limited to, neutral salts, amino acids, peptides of 2 to 5 amino acid residues. In some embodiments, the composition comprises two or more of such agents in combination.

Non-limiting examples of ionic strength modifying agents (v/a) are neutral salts such as sodium chloride, potassium chloride, calcium chloride and magnesium chloride. In one embodiment, the agent (v/a) is sodium chloride.

The term “ionic strength” is the ionic strength of the solution (μ) which is defined by the equation: μ=½ Σ ([i](Zᵢ²)), where μ is the ionic strength, [i] is the millimolar concentration of an ion, and Z₁, is the charge (+ or −) of that ion “(see, e.g., Solomon, Journal of Chemical Education, 78(12):1691-92, 2001; James Fritz and George Schenk: Quantitative Analytical Chemistry, 1979).

In different embodiments of the invention, the ionic strength of the composition is at least 50 mM, such as at least 75 mM, at least 100 mM, at least 150 mM, at least 200 mM, at least 250 mM, at least 400 mM, at least 500 mM, at least 650 mM, at least 800 mM, at least 1000 mM, at least 1200 mM, at least 1600 mM, at least 2000 mM, at least 2400 mM, at least 2800 mM, or at least 3200 mM.

In some specific embodiments, the total concentration of the tonicity modifying agent (v) and the ionic strength modifying agent (v/a) is in the range of 1-1000 mM, such as 1-500 mM, 1-300 mM, 10-200 mM, or 20-150 mM; or such as 100-1000 mM, 200-800 mM, or 500-800 mM, depending on the effect any other ingredients may have on the tonicity and ionic strength.

In one embodiment, the composition is isotonic; in another, it is hypertonic. The term “isotonic” means “isotonic with serum”, i.e. at about 300±50 milliosmol/kg. The tonicity is meant to be a measure of osmolality of the solution prior to administration. The term “hypertonic” is meant to designate levels of osmolality above the physiological level of serum, such as levels above 300±50 milliosmol/kg.

Also, a particular embodiment of the present invention relates to the combination of the stabilising agent (iii) with a fairly high concentration of an ionic strength modifying agent (v/a). In one embodiment thereof, the ionic strength modifying agent (v/a) is selected from the group consisting of sodium salts, calcium salts and magnesium salts. In this embodiment, the ionic strength modifying agent (v/a), i.e. the sodium salt, calcium salt and/or magnesium salt, is present in a concentration of 15-1500 mM, such as 15-1000 mM, 25-1000 mM, 50-1000 mM, 100-1000 mM, 200-1000 mM, 300-1000 mM, 400-1000 mM, 500-1000 mM, 600-1000 mM, 700-1000 mM; 15-800 mM, 25-800 mM, 50-800 mM, 100-800 mM, 200-800 mM, 300-800 mM, 400-800 mM, 500-800 mM; 15-600 mM, 25-600 mM, 50-600 mM, 100-600 mM, 200-600 mM, 300-600 mM; 15-400 mM, 25-400 mM, 50-400 mM, or 100-400 mM.

Within these embodiments, sodium salt may be sodium chloride, the calcium salt may be selected from the group consisting of calcium chloride, calcium acetate, calcium gluconate, and calcium laevulate, and the magnesium salt may be selected from the group consisting of magnesium chloride, magnesium acetate, magnesium gluconate, magnesium laevulate, and magnesium salts of strong acids. In a more specific embodiment, a calcium salt and/or a magnesium salt is/are used in combination with sodium chloride.

In one embodiment, the composition comprises one or more ionic strength modifying agents selected from the group consisting of calcium (Ca²⁺) salts and magnesium (Mg²⁺) salts, e.g. one or more salts selected from the group consisting of calcium chloride, calcium acetate, calcium gluconate, calcium laevulate, magnesium chloride, magnesium acetate, magnesium sulphate, magnesium gluconate, magnesium laevulate, magnesium salts of strong acids.

In one embodiment, the Calcium (Ca²⁺) and/or Magnesium (Mg²⁺) is present in a concentration of at least about 0.1 μM, such as, e.g., at least about 0.5 μM, at least about 1 μM, at least about 5 μM, at least about 10 μM, at least about 50 μM, at least about 100 μM, at least about 1 mM, at least about 2 mM, at least about 5 mM, or at least about 10 mM. In a particular embodiment the composition comprises at least 2 mM Ca²⁺.

In various embodiments, the molar ratio between calcium (Ca²⁺) and/or magnesium ions (Mg²⁺) and FVII polypeptide is: 0.001-750; 0.001-250; 0.001-100; 0.001-10; 0.001-1.0; 0.001-0.5; 0.5-750; 0.5-250; 0.5-100; 0.5-10; 0.5-1.0; 0.001-0.4999; 0.005-0.050.

In one embodiment of the present invention, the molar ratio of non-complexed calcium (Ca²⁺) and/or magnesium (Mg²⁺) to the Factor VII polypeptide is lower than 0.5, e.g. in the range of 0.001-0.499, such as 0.005-0.050, or in the range of 0.000-0.499, such as in the range of 0.000-0.050, or about 0.000. In one embodiment of the present invention, the molar ratio of non-complexed calcium (Ca²⁺) to the Factor VII polypeptide is lower than 0.5, e.g. in the range of 0.001-0.499, such as 0.005-0.050, or in the range of 0.000-0.499, such as in the range of 0.000-0.050, or about 0.000.

When used herein, the term “the concentration of non-complexed calcium and/or magnesium ions” is intended to mean the difference between the total concentration of calcium and/or magnesium ions and the concentration of calcium and/or magnesium bound to calcium/magnesium chelators. In this regard, the Factor VII polypeptide is not regarded as a “calcium/magnesium chelator” although calcium and/or magnesium is expected to bind to, or become associated with, the Factor VII polypeptide under certain conditions.

In another embodiment, the molar ratio of non-complexed calcium and/or magnesium ions to the Factor VII polypeptide is above 0.5. In another embodiment, the molar ratio of non-complexed calcium ions to the Factor VII polypeptide is above 0.5. In order to obtain the low relative ratio between calcium and/or magnesium ions (Ca^{2÷}) and the Factor VII polypeptide, it may be necessary or desirable to remove excess calcium and/or magnesium ions, e.g., by contacting the composition with an ion-exchange material under conditions suitable for removing Ca²⁺ and/or Mg²⁺, or to add a calcium/magnesium chelator in order to bind (complex) excess calcium and/or magnesium ions. This is particularly relevant where the ratio between calcium and/or magnesium ions and the Factor VII polypeptide in a solution from a process step preceding the formulation step exceeds the limit stated above. Examples of “calcium/magnesium chelators” include EDTA, citric acid, NTA, DTPA, tartaric acid, lactic acid, malic acid, succinic acid, HIMDA, ADA and similar compounds.

In a further embodiment, the composition further comprises an antioxidant (vi). In different embodiments, the antioxidant is selected from the group consisting of L-methionine, D-methionine, methionine analogues, methionine-containing peptides, methionine-homologues, ascorbic acid, cysteine, homocysteine, gluthatione, cystine, and cysstathionine. In a preferred embodiment, the antioxidant is L-methionine. The concentration of the antioxidant is typically 0.1-5.0 mg/mL, such as 0.1-4.0 mg/mL, 0.1-3.0 mg/mL, 0.1-2.0 mg/ml, or 0.5-2.0 mg/mL.

In particular embodiments, the composition does not include an antioxidant; instead the susceptibility of the Factor VII polypeptide to oxidation is controlled by exclusion of atmospheric air. The use of an antioxidant may of course also be combined with the exclusion of atmospheric air.

Thus, the present invention also provides an air-tight container (e.g. a vial or a cartridge (such as a cartridge for a pen applicator)) containing a liquid, aqueous pharmaceutical composition as defined herein, and optionally an inert gas. The inert gas may be selected from the group consisting of nitrogen or argon. The container (e.g. vial or cartridge) is typically made of glass or plastic, in particular glass, optionally closed by a rubber septum or other closure means allowing for penetration with preservation of the integrity of the pharmaceutical composition. In a further embodiment, the container is a vial or cartridge enclosed in a sealed bag, e.g. a sealed plastic bag, such as a laminated (e.g. metal (such as aluminium) laminated plastic bag).

In addition to the mandatory components, the non-ionic surfactant (iv), the tonicity modifying agent (v) and the optional antioxidant (vi), the pharmaceutical composition may further comprise a preservative (vii).

A preservative may be included in the composition to retard microbial growth and thereby allow “multiple use” packaging of the Factor VII polypeptides. Examples of preservatives include phenol, benzyl alcohol, orto-cresol, meta-cresol, para-cresol, methyl paraben, propyl paraben, benzalkonium chloride, and benzethonium chloride. The preservative is normally included at a concentration of 0.1-20 mg/mL depending on the pH range and type of preservative.

Still further, the composition may also include one or more agents capable of inhibiting deamidation and isomerisation.

As used herein, pH values specified as “about” are understood to be ±0.1, e.g. about pH 8.0 includes pH 8.0±0.1.

Percentages are (weight/weight) both when referring to solids dissolved in solution and liquids mixed into solutions. For example, for Tween®, it is the weight of 100% stock/weight of solution.

The compositions according to the present invention are useful as stable and preferably ready-to-use compositions of Factor VII polypeptides. Furthermore, it is believed that the principles, guidelines and specific embodiments given herein are equally applicable for bulk storage of Factor VII polypeptides, mutatis mutandis. The compositions are typically stable for at least six months, and preferably up to 36 months; when stored at temperatures ranging from 2° C. to 8° C. The compositions are chemically and/or physically stable, in particular chemically stable, when stored for at least 6 months at from 2° C. to 8° C.

The term “stable” is intended to denote that (i) after storage for 6 months at 2° C. to 8° C. the composition retains at least 50% of its initial biological activity as measured by a one-stage clot assay essentially as described in Assay 3 of the present specification, or (ii) after storage for 6 months at 2° C. to 8° C., the increase in content of heavy chain degradation products is at the most 40% (w/w) of the initial content of Factor VII polypeptide.

The term “initial content” relates to the amount of Factor VII polypeptides added to a composition upon preparation of the composition.

In one embodiment, the stable composition retains at least 70%, such as, e.g., at least 80%, at least 85%, at least 90%, or at least 95%, of its initial biological activity after storage for 6 months at 2 to 8° C.

In different embodiments of the invention, the stable composition further retains at least 50% of its initial biological activity as measured by a one-stage clot assay essentially as described in Assay 3 of the present specification after storage for at least 30 days, such as 60 days or 90 days.

In various embodiments the increase in content of heavy chain degradation products in the stable compositions is not more than about 30% (w/w), not more than about 25% (w/w), not more than about 20% (w/w), not more than about 15% (w/w), not more than about 10% (w/w), not more than about 5% (w/w), or not more than about 3% (w/w) of the initial content of Factor VII polypeptide.

For the purpose of determining the content of heavy chain degradation products, a reverse phase HPLC was run on a proprietary 4.5×250 mm butyl-bonded silica column with a particle size of 5 μm and pore size 300 Å. Column temperature: 70° C. A-buffer: 0.1% v/v trifluoracetic acid. B-buffer: 0.09% v/v trifluoracetic acid, 80% v/v acetonitrile. The column was eluted with a linear gradient from X to (X+13) % B in 30 minutes. X was adjusted so that FVIIa elutes with a retention time of approximately 26 minutes. Flow rate: 1.0 mL/min. Detection: 214 nm. Load: 25 μg FVIIa.

The term “physical stability” of Factor VII polypeptides relates to the formation of insoluble and/or soluble aggregates in the form of dimeric, oligomeric and polymeric forms of Factor VII polypeptides as well as any structural deformation and denaturation of the molecule. Physically stable composition encompasses compositions which remains visually clear. Physical stability of the compositions is often evaluated by means of visual inspection and turbidity after storage of the composition at different temperatures for various time periods. Visual inspection of the compositions is performed in a sharp focused light with a dark background. A composition is classified as physically unstable, when it shows visual turbidity.

The term “chemical stability” is intended to relate to the formation of any chemical change in the Factor VII polypeptides upon storage in solution at accelerated conditions. Examples are hydrolysis, deamidation and oxidation as well as enzymatic degradation resulting in formation of fragments of Factor VII polypeptides. In particular, the sulphur-containing amino acids are prone to oxidation with the formation of the corresponding sulphoxides.

The term “chemically stable” is intended to designate a composition which retains at least 50% of its initial biological activity after storage for 6 months at 2 to 8° C., as measured by a one-stage clot assay (Assay 3).

In a further aspect, the invention also provides a method for preparing a liquid, aqueous pharmaceutical composition of a Factor VII polypeptide, comprising the step of providing the Factor VII polypeptide at a concentration of at least 0.01 mg/mL (i) in a solution comprising a buffering agent (ii) suitable for keeping pH in the range of from about 4.0 to about 9.0; and at least one stabilising agent (iii) comprising a R—CHO motif, wherein R represents aryl, optionally substituted once or several times with hydroxy, C₁₋₆-alkoxy, carboxyl, hydroxymethyl, C₁₋₆-alkyl, C₂₋₆-alkenyl, cyano, or halogen; heteroaryl, optionally substituted once or several times with hydroxy, C₁₋₆-alkoxy, carboxyl, hydroxymethyl, C₁₋₆-alkyl, C₂₋₆-alkenyl, cyano, or halogen; R¹R²R³C—, wherein R¹, R², and R³ independently represent hydrogen, C₁₋₆-alkyl, C₂₋₆-alkenyl, hydroxy, hydroxymethyl, amino, dimethylamino, methoxycarbonylamino, ethoxycarbonylamino, or dimethylaminomethyl; or R⁴C(═O)—, wherein R⁴ represents methyl, ethyl, hydroxy, amino, C₁₋₆-alkoxy, C₁₋₆-alkylamino, or C₂₋₆-dialkylamino.

### Methods of Use

As will be understood, the liquid, aqueous pharmaceutical compositions defined herein can be used in the field of medicine. Thus, the present invention in particular provides the liquid, aqueous pharmaceutical compositions defined herein for use as a medicament, more particular for use as a medicament for treating a Factor VII-responsive disorder.

Consequently, the present invention also provides the use of the liquid, aqueous pharmaceutical composition as defined herein for the preparation of a medicament for treating a Factor VII-responsive disorder, as well as a method for treating a Factor VII-responsive disorder, the method comprising administering to a subject in need thereof an effective amount of the liquid, aqueous pharmaceutical composition as defined herein.

The preparations of the present invention may be used to treat any Factor VII-responsive disorder, such as, e.g., bleeding disorders, including those caused by clotting Factor deficiencies (e.g., e.g. haemophilia A, haemophilia B, coagulation Factor XI deficiency, coagulation Factor VII deficiency); by thrombocytopenia or von Willebrand's disease, or by clotting Factor inhibitors, and intra cerebral haemorrhage, or excessive bleeding from any cause. The preparations may also be administered to patients in association with surgery or other trauma or to patients receiving anticoagulant therapy.

The term “effective amount” is the effective dose to be determined by a qualified practitioner, who may adjust dosages to achieve the desired patient response. Factors for consideration of dose will include potency, bioavailability, desired pharmacokinetic/pharmacodynamic profiles, condition of treatment, patient-related factors (e.g. weight, health, age, etc.), presence of co-administered medications (e.g., anticoagulants), time of administration or other factors known to a medical practitioner.

The term “treatment” is defined as the management and care of a subject, e.g. a mammal, in particular a human, for the purpose of preventing, alleviating or curing a disease or the symptoms of a disease, condition or disorder. This includes the administration of a Factor VII polypeptide to prevent the onset of the symptoms or complications, or alleviating said symptoms or complications, or eliminating the disease, condition, or disorder. Pharmaceutical compositions according to the present invention containing a Factor VII polypeptide may be administered parenterally to subjects in need of such a treatment. Non-exclusive examples of such parenteral administration are subcutaneous, intramuscular or intravenous injection, optionally by means of a pen-like device or an infusion pump.

### EXAMPLES

### General Methods

### Preparation and Purification of Factor VII Polypeptides

Human purified Factor VIIa suitable for use in the present invention is preferably made by DNA recombinant technology, e.g. as described by Hagen et al., Proc.Natl.Acad.Sci. USA 83: 2412-2416, 1986, or as described in European Patent No. 0 200 421 (ZymoGenetics, Inc.).

Factor VII may also be produced by the methods described by Broze and Majerus, J. Biol. Chem. 255 (4): 1242-1247, 1980 and Hedner and Kisiel, J. Clin.Invest. 71: 1836-1841, 1983. These methods yield Factor VII without detectable amounts of other blood coagulation Factors. An even further purified Factor VII preparation may be obtained by including an additional gel filtration as the final purification step. Factor VII is then converted into activated Factor VIIa by known means, e.g. by several different plasma proteins, such as Factor XIIa, IX a or Xa. Alternatively, as described by Bjoern et al. (Research Disclosure, 269 September 1986, pp. 564-565), Factor VII may be activated by passing it through an ion-exchange chromatography column, such as Mono Q® (Pharmacia fine Chemicals) or the like, or by autoactivation in solution.

Factor VII-related polypeptides may be produced by modification of wild-type Factor VII or by recombinant technology. Factor VII-related polypeptides with altered amino acid sequence when compared to wild-type Factor VII may be produced by modifying the nucleic acid sequence encoding wild-type Factor VII either by altering the amino acid codons or by removal of some of the amino acid codons in the nucleic acid encoding the natural Factor VII by known means, e.g. by site-specific mutagenesis.

It will be apparent to those skilled in the art that substitutions can be made outside the regions critical to the function of the Factor VIIa molecule and still result in an active polypeptide. Amino acid residues essential to the activity of the Factor VII polypeptide, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for coagulant, respectively cross-linking activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The introduction of a mutation into the nucleic acid sequence to exchange one nucleotide for another nucleotide may be accomplished by site-directed mutagenesis using any of the methods known in the art. Particularly useful is the procedure that utilizes a super-coiled, double-stranded DNA vector with an insert of interest and two synthetic primers containing the desired mutation. The oligonucleotide primers, each complementary to opposite strands of the vector, extend during temperature cycling by means of Pfu DNA polymerase. On incorporation of the primers, a mutated plasmid containing staggered nicks is generated. Following temperature cycling, the product is treated with DpnI which is specific for methylated and hemi-methylated DNA to digest the parental DNA template and to select for mutation-containing synthesized DNA. Other procedures known in the art for creating, identifying and isolating variants may also be used, such as, for example, gene shuffling or phage display techniques.

Separation of polypeptides from their cell of origin may be achieved by any method known in the art, including, without limitation, removal of cell culture medium containing the desired product from an adherent cell culture; centrifugation or filtration to remove non-adherent cells; and the like.

Optionally, Factor VII polypeptides may be further purified. Purification may be achieved using any method known in the art, including, without limitation, affinity chromatography, such as, e.g., on an anti-Factor VII antibody column (see, e.g., Wakabayashi et al., J. Biol. Chem. 261:11097, 1986; and Thim et al., Biochem. 27:7785, 1988); hydrophobic interaction chromatography; ion-exchange chromatography; size exclusion chromatography; electrophoretic procedures (e.g., preparative isoelectric focusing (IEF), differential solubility (e.g., ammonium sulfate precipitation), or extraction and the like. See, generally, Scopes, Protein Purification, Springer-Verlag, New York, 1982; and Protein Purification, J. C. Janson and Lars Ryden, editors, VCH Publishers, New York, 1989. Following purification, the preparation preferably contains less than 10% by weight, more preferably less than 5% and most preferably less than 1%, of non-Factor VII polypeptides derived from the host cell. Factor VII polypeptides may be activated by proteolytic cleavage, using Factor XIIa or other proteases having trypsin-like specificity, such as, e.g., Factor IXa, kallikrein, Factor Xa, and thrombin. See, e.g., Osterud et al., Biochem. 11:2853 (1972); Thomas, U.S. Pat No. 4,456,591; and Hedner et al., J. Clin. Invest. 71:1836 (1983). Alternatively, Factor VII polypeptides may be activated by passing it through an ion-exchange chromatography column, such as Mono Q® (Pharmacia) or the like, or by autoactivation in solution. The resulting activated Factor VII polypeptide may then be formulated and administered as described in the present application.

### Assays Suitable for Determining the Biological Activity of Factor VII Polypeptides

Factor VII polypeptides useful in accordance with the present invention may be selected by suitable assays that can be performed as simple preliminary in vitro tests.

### In Vitro Hydrolysis Assay (Assay 1)

The in vitro hydrolysis assay is used to assess the ability of Factor VIIa polypeptides to cleave another peptide or protein. The activity measured in the in vitro hydrolysis assay is sometimes referred to as amidolytic activity.

Native (wild-type) Factor VIIa and Factor VIIa polypeptides (both hereinafter referred to as “Factor VIIa”) may be assayed for activities. They may also be assayed in parallel to directly compare their activities. The assay is carried out in a microtiter plate (MaxiSorp, Nunc, Denmark). The chromogenic substrate D-11e-Pro-Arg-p-nitroanilide (S-2288, Chromogenix, Sweden), final concentration 1 mM, is added to Factor VIIa (final concentration 200 nM) in 20 mM imidazole buffer, pH 6.5, containing 20 mM CaCl₂. The absorbance at 405 nm is measured continuously in a SpectraMax™ 340 plate reader (Molecular Devices, USA). The slope of the absorbance developed during a 30-minute incubation period, after subtraction of the absorbance in a blank well containing no enzyme, is used for as a relative measure of Factor VIIa activity

### One-stage Coagulation Assay (Clot Assay) (Assay 3)

The clot assay is used to assess the ability of Factor VIIa polypeptides to make blood clot. For this purpose, the sample to be tested is diluted in 50 mM PIPES-buffer (pH 7.5), 0.1% BSA and 40 μl is incubated with 40 μl of Factor VII deficient plasma and 80 μl of human recombinant tissue factor containing 10 mM Ca2+ and synthetic phospholipids. Coagulation times (clotting times) are measured and compared to a standard curve using a reference standard in a parallel line assay.

### Assays Suitable for Measuring Degradation of Factor VII Polypeptides

Measurement of rFVIIa heavy chain degradation products (Assay 4)

In the below working examples the content of heavy chain fragmentation products of Factor VIIa is determined by RP-HPLC as described in the following:

Reverse phase HPLC was run on a proprietary 4.5×250 mm butyl-bonded silica column with a particle size of 5 μm and pore size 300 Å. Column temperature: 70° C. A-buffer: 0.1% v/v trifluoracetic acid. B-buffer: 0.09% v/v trifluoracetic acid, 80% v/v acetonitrile. The column was eluted with a linear gradient from X to (X+13) % B in 30 minutes. X was adjusted so that FVIIa elutes with a retention time of approximately 26 minutes. Flow rate: 1.0 mL/min. Detection: 214 nm. Load: 25 μg FVIIa.

### Measurement of 40K-PEG-rFVIIa Heavy Chain Fragmentation Products (Assay 5)

For the purpose of determining the content of heavy chain fragmentation products of 40K-PEG-rFVIIa, a reverse phase HPLC was run on a ACE 3 μm C4, 300 Å, 4.6×100 mm column (Advanced Chromatography Technologies, part. no. ACE-213-1046). Column temperature: 60° C. A-buffer: 0.05% v/v trifluoracetic acid. B-buffer: 0.06% v/v trifluoracetic acid, 80% v/v acetonitrile. Denaturation buffer: 6M Guanidine hydrochloride, 50 mM Tris, 5 mM calcium chloride, pH 7,5. Samples are prepared from 50 μl analysis sample+50 μl denaturation buffer+5 μl DTT+1 μl acetic acid and incubated at 60° C. for 15 min.

The column was eluted with a linear gradient from 35 to 80% B in 30 minutes. Flow rate: 0.7 mL/min. Detection: 214 nm. Load: 25 μg FVIIa. The initial content of heavy chain degradation products is subtracted from the measured content of heavy chain degradation product, i.e. the initial content of heavy chain degradation products is set to 0%. The content of heavy chain degradation products at the time x is then calculated as:

$$\begin{matrix}\% = ( HCDP ( x ) - HCDP ( 0 ) ) / ( HCDP ( x ) - HCDP ( 0 ) + FVII ( x ) ) \times 100 \% \\ = ( HCDP ( x ) - HCDP ( 0 ) ) / ( FVII ( 0 ) ) \times 100 \%\end{matrix}$$

wherein HCDP(x) is the measured content of heavy chain degradation products at the time x, HCDP(0) is the measured initial content of heavy chain degradation products, and FVII(x) is the content of the intact Factor VII polypeptide at the time x.

### Worked Examples

The following examples illustrate practice of the invention. These examples are included for illustrative purposes only and are not intended in any way to limit the scope of the invention claimed.

### Example 1

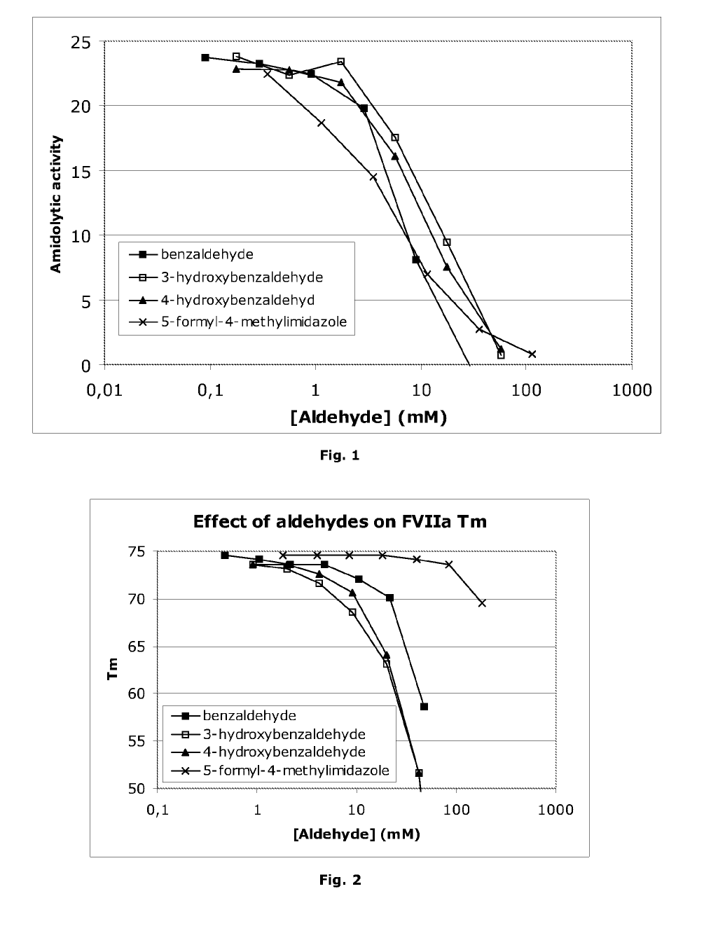

The effect of aldehydes on Factor VIIa amidolytic activity is shown in an in vitro hydrolysis assay (20 mM imidazole, pH 6.5, 20 mM CaCl₂), the results of which are illustrated in FIG. 1. A clear inhibition of amidolytic activity is observed. However, the apparent inhibition could be due to FVIIa denaturation. In order to address this issue, the denaturation temperature of FVIIa was measured in the presence of aldehydes. The measurement was performed by thermo-fluorescence, essentially as the thermal shift assay described by Lo et al., Analytical Biochemistry 332, 153-159 (2004). The denaturation temperature was determined as the point of maximum slope of the fluorescence intensity curve. The denaturation temperature is shown in FIG. 2 at various concentrations of aldehydes (25 mM imidazole, pH 6.5, 20 mM CaCl₂, 60 mg/ml sucrose). In the case of 3-hydroxybenzaldehyde, 4-hydroxybenzaldehyde and benzaldehyde it is seen that the denaturation temperature drops significantly at high aldehyde concentration. Therefore, the inhibitory activity of these three compounds may be caused by denaturation. However, in the case of 5-formyl-4-methylimidazol, the denaturation temperature is unaffected up to a concentration of about 100 mM, suggesting structural integrity of Factor VIIa.

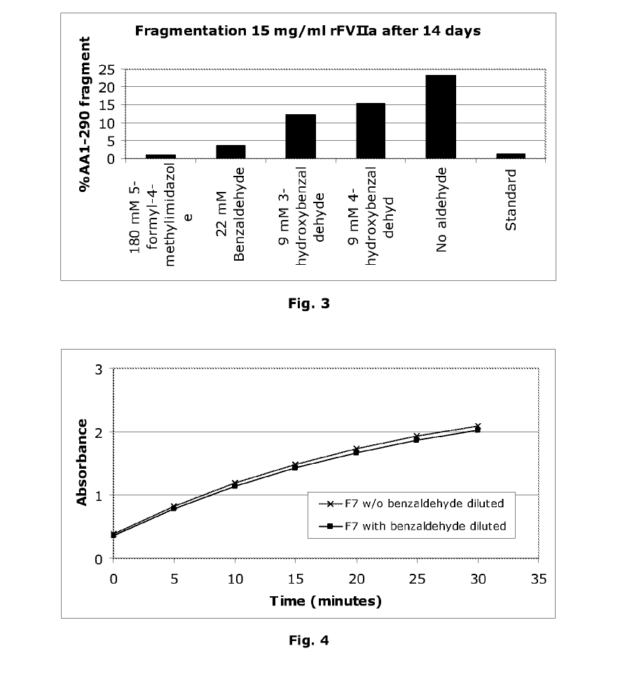

In order to further explore the effect of aldehydes upon FVIIa polypeptides, one degradation product (the 1-290 fragment) was measured after two weeks of storage at 5° C. (in buffer 15 mg/ml rFVIIa, 10 mM imidazol, pH 6,5, 20 mM CaCl2, 24 mg/ml sucrose, 0,2 mg/ml methionine). (NovoSeven® (Novo Nordisk A/S, Denmark) which was analysed immediately after dissolution was used as the “standard”.) The percentage content of the fragment containing a cleavage at position 290 is shown at different aldehyde concentrations in FIG. 3. All of the aldehydes analysed are seen to reduce the amount of FVIIa fragmentation.

To address the important issue of activity recovery, Factor VIIa (17 mg/ml) was incubated with 7 mM benzaldehyde (1 hour, 10 mM imidazol, 35 mM CaCl2, pH 6.5) and then dilution to 0.1 mg/ml Factor VIIa. The amidolytic activity was then measured. FIG. 4 shows the absorbance generated by processing of the subtrate D-11e-Pro-Arg-p-nitroanilide (S-2288, Chromogenix, Sweden) versus time, after dilution of the FVIIa/benzaldehyde solution to 0.1 mg/ml FVIIa (“F7 with benzaldehyde diluted”). The results are compared to the accumulated activity of a diluted FVIIa solution (NovoSeven® (Novo Nordisk A/S, Denmark) which does not contain aldehydes (“F7 w/o benzaldehyde diluted”).

### Example 2

In order to investigate the effect of aldehyde compounds on the stability of rFVIIa, the following formulations are prepared:

Formulation 1:

1.0 mg/mL rFVIIa

10 mM Histidine

10 mM Sodium acetate

10 mM Glycylglycine

50 mM Sodium chloride

10 mM Calcium chloride

50 mM 5-formyl-4-methylimidazol

pH=6.5

Formulation 2:

1.0 mg/mL rFVIIa

10 mM Histidine

10 mM Sodium acetate

10 mM Glycylglycine

50 mM Sodium chloride

10 mM Calcium chloride

50 mM 5-formyl-4-methylimidazol

pH=7.0

Formulation 3:

1.0 mg/mL rFVIIa

10 mM Histidine

10 mM Sodium acetate

10 mM Glycylglycine

50 mM Sodium chloride

10 mM Calcium chloride

pH=6.5

Formulation 4:

1.0 mg/mL rFVIIa

10 mM Histidine

10 mM Sodium acetate

10 mM Glycylglycine

50 mM Sodium chloride

10 mM Calcium chloride

pH=7.0

The formulations are prepared by adding 10 mM histidine, 10 mM sodium acetate and 50 mM 5-formyl-4-methylimidazol (only for formulations 1 and 2) to a 1.0 mg/mL bulk solution of rFVIIa already containing glycylglycine, sodium chloride and calcium chloride in the above mentioned concentrations. pH is finally adjusted to 6.5 and 7.0, respectively, with 1 M sodium hydroxide and 1 M hydrochloric acid.

The formulations are stored at a temperature of 5° C. and 30° C., and analyses for formation of heavy chain degradation products are performed.

### Example 3

Formulation of the following liquid, aqueous pharmaceutical compositions is envisaged:

A)

| | | |
|---|---|---|
| rhFVIIa | 1 | mg/mL (approx. 50,000 IU/mL) |
| PIPES | 15.12 | mg/mL (50 mM) |
| 5-formyl-4-methylimidazol | 10-50 | mM |
| Poloxamer 188 | 0.5 | mg/mL |
| Sodium chloride | 2.92 | mg/mL (50 mM) |
| Calcium chloride 2 H₂O | 1.47 | mg/mL (10 mM) |
| Methionine | 0.5 | mg/mL |

| | |
|---|---|
| 1 M NaOH/1 M HCl | added to pH 6.5 |

B)

| | | |
|---|---|---|
| rhFVIIa | 1 | mg/mL (approx. 50,000 IU/mL) |
| PIPES | 15.12 | mg/mL (50 mM) |
| Benzaldehyde | 10-50 | mM |
| Poloxamer 188 | 0.5 | mg/mL |
| Sodium chloride | 2.92 | mg/mL (50 mM) |
| Calcium chloride 2 H₂O | 1.47 | mg/mL (10 mM) |
| Methionine | 0.5 | mg/mL |

| | |
|---|---|
| 1 M NaOH/1 M HCl | added to pH 6.5 |

C)

| | | |
|---|---|---|
| rhFVIIa | 1 | mg/mL (approx. 50,000 IU/mL) |
| PIPES | 15.12 | mg/mL (50 mM) |
| 3-hydroxybenzaldehyde | 10-50 | mM |
| Poloxamer 188 | 0.5 | mg/mL |
| Sodium chloride | 2.92 | mg/mL (50 mM) |
| Calcium chloride 2 H₂O | 1.47 | mg/mL (10 mM) |

| | |
|---|---|
| 1 M NaOH/1 M HCl | added to pH 6.5 |

D)

| | | |
|---|---|---|
| rhFVIIa | 1 | mg/mL (approx. 50,000 IU/mL) |
| PIPES | 15.12 | mg/mL (50 mM) |
| 4-hydroxybenzaldehyde | 10-50 | mM |
| Poloxamer 188 | 0.5 | mg/mL |
| Sodium chloride | 2.92 | mg/mL (50 mM) |
| Calcium chloride 2 H₂O | 1.47 | mg/mL (10 mM) |

| | |
|---|---|
| 1 M NaOH/1 M HCl | added to pH 6.5 |

Pharmaceutical compositions A-D can subsequently be transferred to sterile vials or cartridges flushed with nitrogen or argon and can then be packed in air-tight aluminium-laminated plastic bags.

### Example 4

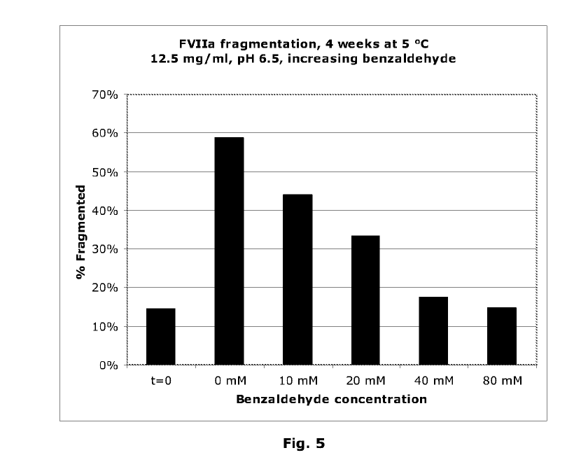

A stability experiment with liquid formulations containing FVIIa was performed. Conditions in all samples were 10 mM His, pH 6.5, 20 mM CaCl₂, 6% sucrose, 0.5 mg/ml methionine, 12.5 mg/ml rFVIIa. 50 μl solutions with 0, 10, 20, 40 and 80 mM benzaldehyde in 500 μl cryotubes were prepared, incubated at 5° C. for 4 weeks and then frozen to −80° C. In addition, a reference sample without benzaldehyde was prepared and immediately frozen to −80° C. For analysis, all samples were thawed, diluted to 1 ml volume in 10 mM histidine, pH 5.5, and analysed for heavy chain fragmentation. FIG. 5 shows the fragmentation as a percentage of the total Factor VIIa content (“% fragmented”). It is clear that fragmentation is much lower in the presence of benzaldehyde, particularly in concentrations above 10 mM.

### Example 5

A stability experiment with liquid formulations containing 40K-PEG-rFVIIa was performed. The formulations had the following compositions:

TABLE 1

| | | | | | Formulation 3 | |
|---|---|---|---|---|---|---|
| Component | Formulation 1 | | Formulation 2 | | (reference) | |
| 40K-PEG-rFVIIa | 20 | mg/ml | 20 | mg/ml | 20 | mg/ml |
| Histidine | 20 | mM | 20 | mM | 20 | mM |
| CaCl₂ | 20 | mM | 20 | mM | 20 | mM |
| Methionine | 0.5 | mg/ml | 0.5 | mg/ml | 0.5 | mg/ml |
| Sucrose | 60 | mg/ml | 60 | mg/ml | 60 | mg/ml |

| | | | | |
|---|---|---|---|---|
| Benzaldehyde | 28 | mM | — | — |

| | | | | |
|---|---|---|---|---|
| 5-formyl-4- | — | 180 | mM | — |
| methylimidazole | | | | |

The PEG moiety is excluded in the specification of 40K-PEG-rFVIIa concentration. pH was 6.5 in all samples. At 0, ½, 1 and 2 months, 70 μl aliquots were taken from the stability sample, diluted to 1 mg/ml 40K-PEG-rFVIIa and assayed for clot activity (Δactivity), FVIIa polypeptide fragmentation (Δfragment), dimer/2-PEG, and high molecular weight protein. None of the samples showed any increase in dimer/2-PEG or high molecular weight protein. Table 2 shows the change in FVII polypeptide fragmentation and relative change in clot activity after 2 months at 5° C. for the three formulations:

TABLE 2

| Formulation | Δfragment | Δactivity |
|---|---|---|
| 1 | +29.3% | −59% |
| 2 | +1.5% | −2% |
| 3 | +53.3% | −67% |

The results show that the addition of 28 mM benzaldehyde results in a small reduction in fragmentation and loss of activity, when compared to the reference formulation. 180 mM 5-formyl-4-methylimidazole, on the other hand, gives a very large reduction in fragmentation and a very large improvement in FVIIa polypeptide stability.

1. A liquid, aqueous pharmaceutical composition comprising
(i) at least 0.01 mg/mL of a Factor VII polypeptide;
(ii) a buffering agent suitable for keeping pH in the range of from about 5.0 to about 7.0; and
(iii) at least one stabilising agent comprising a R—CHO motif, wherein R represents aryl, optionally substituted once or several times with hydroxy, C₁₋₆-alkoxy, carboxyl, hydroxymethyl, C₁₋₆-alkyl, C₂₋₆-alkenyl, cyano, or halogen; heteroaryl, optionally substituted once or several times with hydroxy, C₁₋₆-alkoxy, carboxyl, hydroxymethyl, C₁₋₆-alkyl, C₂₋₆-alkenyl, cyano, or halogen; R¹R²R³C—, wherein R¹, R², and R³ independently represent hydrogen, C₁₋₆-alkyl, C₂₋₆-alkenyl, hydroxy, hydroxymethyl, amino, dimethylamino, methoxycarbonylamino, ethoxycarbonylamino, or dimethylaminomethyl; or R⁴C(═O)—, wherein R⁴ represents methyl, ethyl, hydroxy, amino, C₁₋₆-alkoxy, C₁₋₆-alkylamino, or C₂₋₆-dialkylamino.

2. The composition according to claim 1, wherein R represents aryl, optionally substituted once or several times with hydroxy, methoxy, carboxyl, hydroxymethyl, methyl, ethyl, isopropyl, or cyano; heteroaryl, optionally substituted once or several times with hydroxy, methoxy, carboxyl, hydroxymethyl, methyl, ethyl, isopropyl, or cyano; R¹R²R³C—, wherein R¹, R², and R³ independently represent hydrogen, methyl, hydroxy, hydroxymethyl, amino, dimethylamino, methoxycarbonylamino, ethoxycarbonylamino, or dimethylaminomethyl; or R⁴C(═O)—, wherein R⁴ represents methyl, ethyl, hydroxy, amino, methoxy, ethoxy, methylamino or dimethylamino.

3. The composition according to claim 1, wherein R represents phenyl, optionally substituted once or several times with hydroxy, carboxyl, or hydroxymethyl; imidazolyl, optionally substituted once or several times with hydroxy, carboxyl, hydroxymethyl, or methyl; R¹R²R³C, wherein R¹, R², and R³ independently represent hydrogen, methyl, hydroxy, hydroxymethyl, methoxycarbonylamino, ethoxycarbonylamino, or dimethylaminomethyl; or R⁴C(═O)—, wherein R⁴ represents methyl, ethyl, hydroxy, amino, methoxy, ethoxy, methylamino or dimethylamino.

4. The composition according to claim 1, wherein the stabilising agent (iii) is at least one selected from the group consisting of benzaldehyde, acetaldehyde, pivalic aldehyde (trimethylacetaldehyde), isobutyraldehyde, 4-methyl-5-formylimidazole, 3-hydroxybenzaldehyde, hydroxypivaldehyde, 3-dimethylamino-2,2-dimethylpropionaldehyde, salicylaldehyde, 4-hydroxybenzaldehyde, 2,3-dihydroxybenzaldehyde, 2,4-dihydroxybenzaldehyde, 2,5-dihydroxybenzaldehyde, 3,4-dihydroxybenzaldehyde, 2-carboxybenzaldehyde, 4-carboxybenzaldehyde, 2-formylpyridine, 3-formylpyridine, 4-formylpyridine, 2-formylimidazole, 4-formylimidazole, and 2-hydroxymethyl-4-formylimidazole.

5. The composition according to claim 1, wherein the concentration of the stabilising agent (iii) is at least 1 μM, such as at least 1 mM; such as at least 2 mM.

6. The composition according to claim 1, wherein the Factor VII polypeptide is human Factor VII(a).

7. The composition according to claim 1, wherein the Factor VII polypeptide is PEGylated or glycoPEGylated.

8. The composition according to claim 1, wherein the Factor VII polypeptide is a Factor VII(a) sequence variant.

9. The composition according to claim 8, wherein the ratio between the activity of the Factor VII polypeptide and the activity of native human Factor VIIa (wild-type FVIIa) is at least 1.25 when tested in the “In Vitro Proteolysis Assay” (Assay 2) as described herein.

10. The composition according to claim 1, wherein the Factor VII polypeptide is present in a concentration of about 0.01-30.0 mg/mL.

11. The composition according to claim 1, which has a pH value in the range of from about 5.0 to about 7.0; such as from about 5.0 to about 6.5; such as from about 5.0 to about 6.0; such as from about 5.5 to about 7.0; such as from about 5.5 to about 6.5; such as from about 6.0 to about 7.0; such as from about 6.0 to about 6.5; such as from about 6.3 to about 6.7; or from about 5.2 to about 5.7.

12. The composition according to claim 1, wherein the buffering agent (ii) comprises at least one component selected from the group consisting of acids and salts of MES, PIPES, ACES, BES, TES, HEPES, TRIS, histidine, imidazole, glycine, glycylglycine, glycinamide, phosphoric acid, acetic acid, lactic acid, glutaric acid, citric acid, tartaric acid, malic acid, maleic acid, and succinic acid.

13. The composition according to claim 12, wherein the concentration of the buffering agent (ii) is 1-100 mM.

14. The composition according to claim 7, wherein (iii) is 5-formyl-4-methylimidazole in a concentration of about 0.5-200 mM.

15. The composition according to claim 7, wherein (iii) is benzaldehyde in a concentration of about 0.5-100 mM.
